(19) United States
(12) Patent Application Publication
(10) Pub. No.: US 2022/0047755 A1
(43) Pub. Date: Feb. 17, 2022

(54) METHOD OF OPERATING A FRAGRANCING DEVICE

(71) Applicant: MAHLE International GmbH, Stuttgart (DE)

(72) Inventors: Thomas FEITH, Pforzheim (DE); Andre BAUER, Wendlingen (DE); Christian SCHEURER, Leinfelden-Echterdingen (DE)

(73) Assignee: MAHLE International GmbH, Stuttgart (DE)

(21) Appl. No.: 17/401,852

(22) Filed: Aug. 13, 2021

(30) Foreign Application Priority Data
Aug. 13, 2020 (DE) 10 2020 210 305.4

(51) Int. Cl. A61L 9/12; B60H 3/00

(52) U.S. Cl. CPC A61L 9/122; B60H 3/0021; B60H 2003/0042; A61L 2209/11; A61L 2209/134; B60H 3/0035

(57) ABSTRACT

A method for operating a fragrancing device, wherein a blower device is provided which sucks air through an air inlet opening into the first air duct and conveys it to an air outlet opening. At second air duct is provided, which branches off from the first air duct at a first duct branch and rejoins the first air duct at a second duct branch. A first fragrance container is provided in the second air duct between the first duct branch and the second duct branch, which can be flowed through by the air flow in the second air duct. A first valve device is provided upstream and/or downstream and controls the flow through the first fragrance container. The blower is controlled in such a manner that a defined, substantially constant, air flow is generated as a sum through the first air duct and the second air duct.

This nonprovisional application claims priority under 35 U.S.C. § 119(a) to German Patent Application No. 10 2020 210 305.4, which was filed in Germany on Aug. 13, 2020, and which is herein incorporated by reference.

### BACKGROUND OF THE INVENTION

### Field of the Invention

The present invention relates to a method for operating a fragrancing device, in particular for fragrancing a vehicle interior.

### Description of the Background Art

A variety of fragrancing devices are known in the state of the art. For example, passive fragrancing devices are known which inherently release fragrances into the surrounding air, wherein in the case of passive fragrancing devices, the release of the fragrance is not controlled.

Fragrancing devices have also become known in which the fragrance is discharged into an air flow. Typically, the fragrancing device discharges the fragrance into an air flow in an air duct in a metered manner, wherein the quantity of fragrance delivered is determined and delivered in a controlled manner in order to meter the intensity of the fragrance. The quantity of air to which the fragrance is dispensed is not particularly important.

It has been shown with such fragrancing devices that the fragrancing of the entire vehicle interior occurs only very inhomogeneously, in particular the passengers in a rear row of seats, in the back, often only notice the fragrance in very small doses.

### SUMMARY OF THE INVENTION

It is therefore an object of the present invention to provide a method for operating a fragrancing device in which a more uniform and, in particular, stronger fragrancing of a vehicle interior is made possible.

An exemplary embodiment of the invention relates to a method for operating a fragrancing device, wherein the fragrancing device has a first air duct which has an air inlet opening and an air outlet opening, wherein a blower device is provided which sucks or blows air through the air inlet opening into the first air duct and conveys it to the air outlet opening, wherein at least one second air duct is provided, which branches off from the first air duct at a first duct branch and rejoins the first air duct at a second duct branch, wherein a first fragrance container is provided in the second air duct between the first duct branch and the second duct branch, which can be flowed through by the air flow in the second air duct, wherein a first valve device is provided upstream and/or downstream of the first fragrance container, which valve device controls the flow through the first fragrance container, wherein the blower is controlled in such a manner such that a defined, substantially constant, air flow is generated as a sum through the first air duct and the second air duct, the degree of fragrancing being controlled by the controlled outputted quantity of fragrance to the air flow in the second air duct and/or by the quantity of air of the air flow in the second air duct. As a result, the air quantity remains substantially permanently at an equal and, in particular, high level, which air is fragranced and discharged from the fragrancing device, so that the air quantity is sufficient to flow through and fragrance the vehicle interior as completely as possible.

It is also advantageous if a third air duct is provided, which branches off from the first air duct at a third duct branch and rejoins the first air duct at a fourth duct branch, wherein a second fragrance container is provided in the third air duct between the third duct branch and the fourth duct branch, through which second fragrance container the air flow in the third air duct can flow, wherein a second valve device is provided upstream and/or downstream of the second fragrance container, which second valve device controls the flow through the second fragrance container. Thus, depending on the flow through the first and/or the second fragrance container, the outflowing fragrance can be selected in a user-specific manner.

It is also particularly advantageous if the blower is controlled in such a way that a defined, essentially constant, air flow is generated through the first air duct and the second air duct and/or the third air duct, i.e. an essentially constant air flow as a sum through the fragrancing device, wherein the degree of fragrancing is controlled by the controlled outputted quantity of fragrance to the air flow in the second air duct and/or the third air duct and/or by the air quantity of the air flow in the second air duct and/or in the third air duct. Thus, reliable fragrancing of the interior of the motor vehicle can be achieved.

It is also advantageous if the third air duct branches off from the first air duct directly or indirectly and rejoins the first air duct directly or indirectly. Thus, a further design of a fragrancing device can be achieved, which achieves reliable fragrancing.

It is also advantageous if the third air duct branches off indirectly from the first air duct by branching off from the second air duct, and/or if the third air duct rejoins the first air duct directly indirectly by joining the second air duct. This way, a further fragrancing device design is obtained, which achieves reliable fragrancing.

For reliable fragrancing, it is also advantageous if the air flow enriched with fragrance through the second air duct and/or the air flow enriched with fragrance through the third air duct is mixed with the air flow through the first air duct. Thus, a fragrance-charged air flow is generated through the second and/or the third air duct, which is generated as a bypass flow to the air flow through the first air duct and is admixed to the air flow through the first air duct, so that altogether an air flow with a high air volume results, which air flow is loaded with the fragrance and which air flow, high in sum total and with a high air volume, however, does not altogether have to flow through the fragrance container(s), which is advantageous with regard to noise and pressure drop. The partial air flow that flows through the fragrance container(s) can be low in terms of air volume, although the total air flow can have a high air volume.

It is also advantageous if the air outlet opening is subdivided into partial air outlet openings and the air flow enriched with fragrance is distributed to at least individual, several or all of the partial air outlet openings. In this way, a better distribution of the air flow laden with air and thus a better distribution of the fragrance can be achieved.

It is also advantageous if the air outlet direction of the air outlet opening or the partial air outlet openings is/are different, variable or variably adjustable, so that the air flow enriched with fragrance can be discharged in different spatial directions. This can also be used to achieve a better distribution of the air flow laden with air and thus a better distribution of the fragrance.

It is also advantageous if the air outlet directions of at least individual or several or all partial air outlet openings are set in a different direction. This can also achieve a better distribution of the air flow laden with air and thus a better distribution of the fragrance.

It is also advantageous if the partial air outlet openings are distributed in the vehicle interior, in particular in the front area and in the rear area, such as in particular in the area of the instrument panel, the mirror base and/or the headliner of the motor vehicle.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 is a schematic representation of a fragrancing device for carrying out the method according to the invention, and

FIG. 2 is a representation of a variant of the fragrancing device according to FIG. 1.

### DETAILED DESCRIPTION

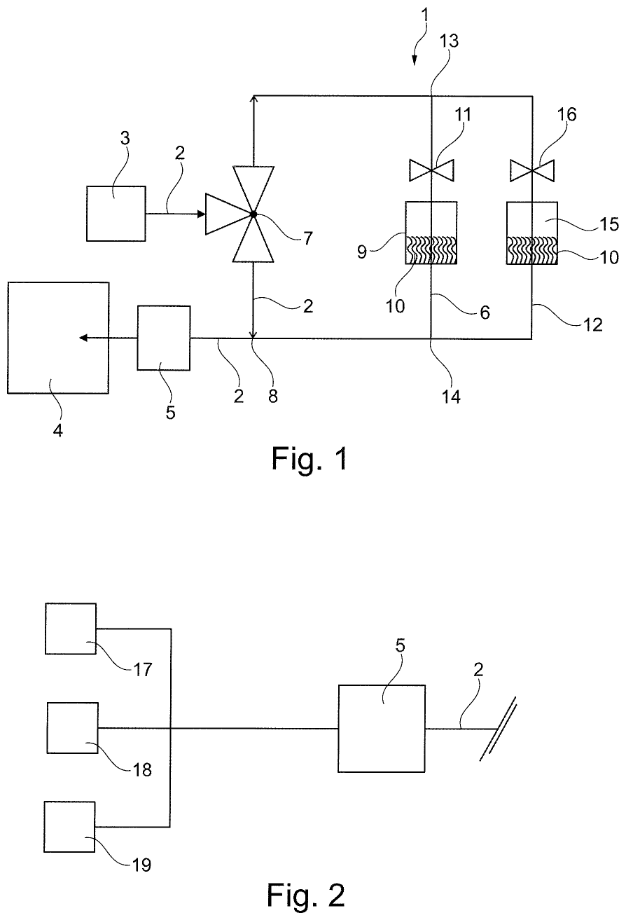

FIG. 1 shows a schematic representation of a fragrancing device 1.

The fragrancing device 1 has a first air duct 2, which has an air inlet opening 3 and an air outlet opening 4. Thus, air can enter the first air duct 2 through the air inlet opening 3 and the air can be discharged again through the air outlet opening 4.

A blower device 5 is provided to control the flow, which sucks or blows air through the air inlet opening 3 into the first air duct 2 and conveys it towards the air outlet opening 4. The blower 5 may be, for example, a spiral blower.

Also provided is at least one second air duct 6, which branches off from the first air duct 2 at a first duct branch 7 and rejoins the first air duct 2 at a second duct branch 8.

In the second air duct 6, a first fragrance container 9 is provided between the first duct branch 7 and the second duct branch 8, through which the air flow in the second air duct 6 can flow. As a result, a quantity of the fragrance 10 can be delivered from the fragrance container 9 to the air flowing through the fragrance container 9 so that the air absorbs and carries away the fragrance 10 to fragrance a vehicle interior of a motor vehicle.

A first valve device 11 is provided upstream and/or downstream of the first fragrance container 9 for controlling the flow through the fragrance container 9 and thus also the delivery of the fragrance 10 to the air flowing through. In FIG. 1, the first valve device 11 is arranged upstream of the fragrance container 9. Alternatively, or additionally, a valve device may also be arranged downstream of the fragrance container 9.

The method of operating the fragrancing device 1 according to the invention provides that the blower 5 is controlled in such a way that a defined, substantially constant, air flow is generated as a sum through the first air duct 2 and the second air duct 6, wherein the degree of fragrancing is controlled by the controlled outputted quantity of fragrance to the air flow in the second air duct 6 and/or by the quantity of air of the air flow in the second air duct 6. The first air duct 2 and the second air duct 6 are quasi-connected in parallel when the valve device 11 is open so as to produce a defined total air flow.

According to a further development of the fragrancing device 1, a third air duct 12 can also be provided, which branches off from the first air duct 2 at a third duct branch 13 and rejoins the first air duct 2 at a fourth duct branch 14, wherein a second fragrance container 15 is provided in the third air duct 12 between the third duct branch 13 and the fourth duct branch 14, through which container the air flow in the third air duct 12 can flow.

In this case, the third air duct 12 can branch off from the first air duct 2 directly or indirectly and/or rejoin the first air duct 2 directly or indirectly.

For example, the third air duct 12 may branch off indirectly from the first air duct 2 by branching off from the second air duct 6 and/or the third air duct 12 may rejoin the first air duct 2 indirectly by uniting with the second air duct 6, also see FIG. 1.

Advantageously, a second valve device 16 is provided upstream and/or downstream of the second fragrance container 15, which controls the flow through the second fragrance container 15. In FIG. 1, the second valve device 16 is arranged upstream of the second fragrance container 15. Alternatively, or additionally, a valve device can also be arranged downstream of the second fragrance container 15.

According to the invention, the blower 5 can be controlled in such a way that a defined, in particular substantially constant, air flow is generated through the first air duct 2 and/or the second air duct 6 and/or the third air duct 12, wherein the degree of fragrancing is controlled by the controlled outputted quantity of fragrance to the air flow in the second air duct 6 and/or the third air duct 12 and/or by the air quantity of the air flow in the second air duct 6 and/or in the third air duct 12.

In this case, the air flow enriched with fragrance through the second air duct 6 and/or the air flow enriched with a fragrance 10 through the third air duct 12 is mixed with the air flow through the first air duct 2. The total air flow remains essentially unchanged.

FIG. 2 shows a variation of FIG. 1, in which the air outlet opening 4 of FIG. 1 is divided into partial air outlet openings 17, 18, 19 in FIG. 2 and the air flow enriched with fragrance 10 is distributed to at least one, several or all of the partial air outlet openings 17, 18, 19. For this purpose, an air flow control element and/or valve element can be provided for each partial air outlet opening 17, 18, 19 or for each group of partial air outlet openings 17, 18, 19.

Thereby, the air outlet direction of the air outlet opening 4 or the partial air outlet openings 17, 18, 19 can be different, variable or variably adjustable, so that the air flow enriched with fragrance 10 can be discharged in different spatial directions. In this case, the air enriched with fragrance 10 can be discharged simultaneously in different spatial directions or time-dependently, so that the pivoting of the spatial direction takes place as a function of time.

Also, the air outlet direction of at least individual or several or all partial air outlet openings 17, 18, 19 can each be set or adjusted in a different direction.

The partial air outlet openings 17, 18, 19 can also be distributed in the vehicle interior, in particular in the front area and in the rear area, such as in particular in the area of the instrument panel, the mirror base and/or the headliner of the motor vehicle.

The fragrancing device 1 can be designed as a self-sufficient device, which is not designed as an integrated component of the motor vehicle, but which can be operated and arranged independently. There may be an independent power supply by means of batteries or rechargeable batteries, or an electrical supply connection to the motor vehicle electrical system may be provided.

Alternatively, the fragrancing device 1 could also be part of the vehicle equipment.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A method for operating a fragrancing device, wherein the fragrancing device has a first air duct which has an air inlet opening and an air outlet opening, the method comprising:
sucking or blowing air via a blower device through the air inlet opening into the first air duct and conveying the air the air outlet opening;
providing at least one second air duct, which branches off from the first air duct at a first duct branch and rejoins the first air duct at a second duct branch;
providing a first fragrance container in the second air duct between the first duct branch and the second duct branch, which is adapted to be flowed through by the air flow in the second air duct;
controlling the flow through the first fragrance container via a first valve device upstream and/or downstream of the first fragrance container;
controlling the blower such that a defined or essentially constant air flow is generated as a sum through the first air duct and the second air duct; and
controlling a degree of fragrancing by the controlled outputted quantity of fragrance to the air flow in the second air duct and/or by the air quantity of the air flow in the second air duct.

2. The method according to claim 1, wherein a third air duct is provided, which branches off from the first air duct at a third duct branch and rejoins the first air duct at a fourth duct branch, wherein a second fragrance container is provided in the third air duct between the third duct branch and the fourth duct branch, which is adapted to be flowed through by the air flow in the third air duct, wherein a second valve device is provided upstream and/or downstream of the second fragrance container, which controls the flow through the second fragrance container.

3. The method according to claim 2, wherein the blower is controlled such that a defined or substantially constant air flow is generated through the first air duct and the second air duct and/or the third air duct, wherein the degree of fragrancing is controlled by the controlled outputted quantity of fragrance to the air flow in the second air duct and/or the third air duct and/or by the air quantity of the air flow in the second air duct and/or in the third air duct.

4. The method according to claim 2, wherein the third air duct branches off from the first air duct directly or indirectly and rejoins the first air duct directly or indirectly.

5. The method according to claim 4, wherein the third air duct branches off indirectly from the first air duct by branching off from the second air duct and/or the third air duct rejoins the first air duct indirectly by joining the second air duct.

6. The method according to claim 1, wherein the air flow enriched with fragrance through the second air duct and/or the air flow enriched with a fragrance through the third air duct is admixed with the air flow through the first air duct.

7. The method according to claim 1, wherein the air outlet opening is divided into partial air outlet openings and the air flow enriched with fragrance is distributed to at least individual, several or all of the partial air outlet openings.

8. The method according to claim 1, wherein the air outlet direction of the air outlet opening or the partial air outlet openings is/are different, variable or variably adjustable, so that the air flow enriched with fragrance is adapted to be discharged in different spatial directions.

9. The method according to claim 8, wherein the air outlet directions of at least individual or several or all partial air outlet openings are set in a different direction in each case.

10. The method according to claim 7, wherein the partial air outlet openings are arranged distributed in the vehicle interior, in particular in the front region and in the rear region, such as in particular in the region of the instrument panel, the mirror base and/or the headliner of the motor vehicle.
